(19) United States
(12) Patent Application Publication
(10) Pub. No.: US 2006/0122505 A1
(43) Pub. Date: Jun. 8, 2006

(54) M-Mode presentation of an ultrasound scan

(75) Inventor: Praveen Dala-Krishna, Bensalem, PA (US)

Correspondence Address: HELLER EHRMAN WHITE & MCAULIFFE LLP, 1717 RHODE ISLAND AVE, NW, WASHINGTON, DC, 20036-3001, US

(73) Assignee: EP MedSystems, Inc., West Berlin, NJ (US)

(21) Appl. No.: 10/994,425

(22) Filed: Nov. 23, 2004

(51) Int. Cl. A61B 8/00

(52) U.S. Cl. 600/437

(57) ABSTRACT

A method and apparatus for processing ultrasound images is provided, including extracting an M-mode data set from the ultrasound images, discerning, from the M-mode data set, a first velocity for a first moving structure and a second velocity for a second moving structure, the second velocity being different from the first velocity, and differentiating between the first moving structure and the second moving structure based on the discerned velocities.

### BACKGROUND OF THE INVENTION

1. Field of the Invention

The present invention relates generally to medical imaging systems, and more particularly to a method and apparatus for m-mode presentation of an ultrasound scan.

2. Description of the Related Art

Ultrasound devices have been developed and refined for the diagnosis and treatment of various medical conditions. Such devises have been developed, for example, to track the magnitude and direction of motion of moving objects, and/or the position of moving objects over time. By way of example, Doppler echocardiography is one ultrasound technique used to determine motion information from the recording and measurement of Doppler data for the diagnosis and treatment of cardiac conditions, and is described in greater detail below.

The Doppler principle, as used in Doppler echocardiography, generally involves exploiting an observed phenomenon that the frequency of reflected ultrasound pulses is altered by a moving object, such as moving tissue or blood cells. This alteration is generally referred to as a Doppler shift. The magnitude of the Doppler shift relates to the velocity of the moving object, and the polarity of the Doppler shift (positive=towards, negative=away) relates to the direction of motion relative to the ultrasound source. As such, the magnitude and polarity of the Doppler shift can be used to track the magnitude and direction of motion of moving objects.

Treatment and diagnosis techniques operating on the Doppler principle generally involve one of three Doppler modalities, continuous wave (CW) Doppler, pulsed wave (PW) Doppler, and color Doppler. The selection of CW Doppler or PW Doppler for a particular application depends on the requirements of the particular application at hand, as each technique has features and limitations readily apparent to those of skill in the art. In regards to color Doppler, this technique generally involves the addition of regions of interest to a PW Doppler based scan, each region of interest being superimposed with a color scale based on velocity, direction of motion, etc. As such, color Doppler can be thought of as an enhanced PW Doppler scan. The aforementioned Doppler modalities have been applied to the diagnosis and treatment of cardiac conditions, and can be grouped together and referred to generally as echocardiography.

Another group of ultrasound imaging techniques include a class generally referred to as brightness mode (“B-Mode”) displays. To generate a B-Mode display, an ultrasound pulse and its echo are measured and used to determine the distance of a given object from the ultrasound transducer, and the signal intensity at that distance. A display is then rendered from a collection of the ultrasound data, where the position of each “dot” corresponds to the distance from the ultrasound transducer of a given object, and the brightness of each “dot” corresponds to the signal strength at that position.

Yet another class of ultrasound imaging techniques is generally referred to as motion mode (“M-Mode”) displays. To generate an M-Mode display, a time interval between a first ultrasound pulse and the echo in response thereto (which corresponds to depth) is plotted along one axis. Subsequent time intervals for subsequent ultrasound pulses (and their corresponding echoes) are then plotted along the other axis (which corresponds to time). This plot graphically depicts movement of a given object over time. Such a technique is described in U.S. Pat. No. RE37,088, which is incorporated by reference herein in its entirety.

The aforementioned ultrasound imaging techniques have given clinicians a wide variety of tools with which to diagnose and treat various medical conditions, such as the noted cardiac conditions. These tools are limited, however, in their ability to discern between various structures, and their ability to accurately track (and display) a moving structure amongst a plurality of moving structures. Thus, a need exists for an improved method of processing ultrasound images.

Other problems with the prior art not described above can also be overcome using the teachings of the present invention, as would be readily apparent to one of ordinary skill in the art after reading this disclosure.

### SUMMARY OF THE INVENTION

According to an embodiment of the present invention, a method of processing ultrasound images is provided, including extracting an M-mode data set from the ultrasound images, discerning, from the M-mode data set, a first velocity for a first moving structure and a second velocity for a second moving structure, the second velocity being different from the first velocity, and differentiating between the first moving structure and the second moving structure based on the discerned velocities.

According to an embodiment of the present invention, a method of processing ultrasound images is provided, including extracting an M-mode data set from the ultrasound images, discerning a cyclical motion of a moving structure, and tracking the moving structure having the discerned cyclical motion.

According to an embodiment of the present invention, a method of processing ultrasound images is provided, including extracting an M-mode data set from the ultrasound images, discerning, from the M-mode data set, a velocity for a moving structure, tracking the discerned velocity for the moving structure over time, and identifying the moving structure on the basis of the tracked velocity.

According to an embodiment of the present invention, an ultrasound imaging device is provided, including an interface adapted and configured to receive ultrasound image data, and a controller. The controller is adapted and configured to extract an M-mode data set from the ultrasound image data, discern, from the M-mode data set, a first velocity for a first moving structure and a second velocity for a second moving structure, the second velocity being different from the first velocity, and differentiate between the first moving structure and the second moving structure based on the discerned velocities.

According to an embodiment of the present invention, an ultrasound imaging system is provided, including means for acquiring ultrasound images of a region, means for extracting an M-mode data set from the ultrasound images, and means for differentiating between a first moving structure and a second moving structure in the region based on differences between the velocity and/or direction of motion of the first moving structure and the second moving structure.

### BRIEF DESCRIPTION OF THE DRAWINGS

FIG. 1 depicts an exemplary imaging system usable with various embodiments of the present invention.

FIG. 2 depicts a method of processing ultrasound images according to an embodiment of the present invention.

FIG. 3 depicts a method of tracking moving structures according to an embodiment of the present invention.

FIG. 4 depicts a method of identifying moving structures according to an embodiment of the present invention.

### DETAILED DESCRIPTION OF THE EXEMPLARY EMBODIMENTS

Reference will now be made in detail to exemplary embodiments of the present invention. Wherever possible, the same reference numbers will be used throughout the drawings to refer to the same or like parts.

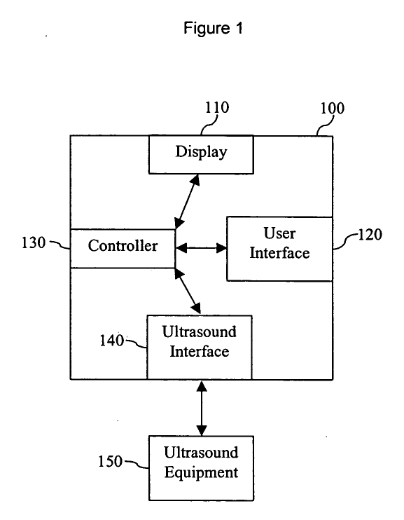

An exemplary imaging system usable with various embodiments of the present invention is shown in the block diagram of FIG. 1. The imaging system includes an ultrasound imaging device 100, such as a workstation, in electrical communication with ultrasound equipment 150. The ultrasound imaging device 100 preferably includes a display 110, a user interface 120, and an ultrasound interface 140 all electrically coupled to controller 130. According to one aspect of the present invention, controller 130 comprises an appropriately programmed microprocessor, an application specific integrated circuit (ASIC), or other suitable device. Additional components may be provided as would be readily apparent to one of ordinary skill in the art after reading this disclosure.

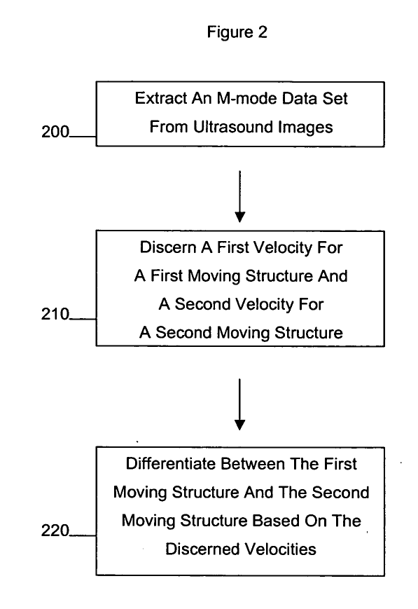

A method of processing ultrasound images according to an embodiment of the present invention is shown in the flowchart of FIG. 2. It should be appreciated that, as with other methods to be described below, the method shown in FIG. 2 may be implemented using the exemplary imaging system of FIG. 1, or using another suitable imaging system.

As shown in FIG. 2, in step 200 the imaging system extracts an M-mode data set from a plurality of ultrasound images. By way of example, step 200 may be performed using the techniques disclosed in RE37,088, or using another suitable technique. In step 210, the imaging system discerns, based on the M-mode data set extracted in step 200, a first velocity for a first moving structure and a second velocity for a second moving structure, the second velocity preferably being different from the first velocity by a discernable amount. While step 210 describes determining only two velocities, it should be appreciated that more than two velocity determinations can be made in step 210 (e.g., for data points along a line of interest in the M-mode data set). Such determinations may be made, for example, using the Doppler principles previously described, for transmitting, receiving and processing signals from various points along the line of interest to allow processing of Doppler data. This processing of Doppler data allows for estimating the velocity of a structure or blood at the given point.

Where a group of data points have determined velocities within a given range from each other (e.g., velocities not more than ±1%, 5%, 10%, etc. from a median/mean velocity), the image processing device may consider these to be different portions of the same moving structure. According to another example, where a difference in velocity greater than or equal to a known value is determined in step 210, the image processing device may consider this to represent a boundary between two structures. In either of these conditions, this data can thus be used to differentiate between moving structures in step 220, as illustrated by the following hypothesis:

Assume three regions with corresponding data groups—(1) a first group of data points within a heart leading up to a heart wall; (2) a second group of data points within the heart wall itself; and (3) a third set of data points outside of the heart wall and external to the heart. The first group of data points may have a first velocity, such as a velocity of blood moving within the heart near the heart wall. The second group of data points may have a second velocity, representing movement of the heart wall itself (e.g., a heart contraction causing the heart wall to move with respect to the ultrasound transducer). The third group of data points may have a third velocity, such as zero velocity (e.g., representing no movement of the region outside the heart wall). These velocities are first determined in step 210, once the M-mode data set has been extracted in step 200.

According to one embodiment of the present invention, the imaging system may discern the first region, the second region, and the third region from each other in step 220 by identifying the similarity in velocities of each group. As an example, if first group has a first velocity of about X, the second group a second velocity of about Y, and the third group a third velocity of about Z with each data point in the first, second and third groups being ±a known value K from X, Y and Z respectively, the imaging system may identify X, Y and Z and group the data points into the first, second and third groupings. Each grouping thus represents a different moving structure, as a given moving structure will have an average velocity (X, Y or Z) generally constant or vary in known manner throughout a portion of the cardiac cycle.

Alternatively, the imaging system may discern the first region, the second region, and the third region from each other by identifying a change in velocity greater than or equal to a known value K. By way of example, the imaging system may discern a boundary exists between the first group and the second group by identifying that X velocity and Y velocity are different by at least K, and that a boundary exists between the second group and the third group by identifying that Y velocity and Z velocity are different by at least K. Thus, where a sharp enough transition in velocity (represented by K) exists, a structural boundary exists. This would be in addition to any variation in brightness/amplitude of the backscattered ultrasound signal from the regions corresponding to X and Y and their relative spatial separations. Each grouping thus represents a different moving structure, as a given moving structure will have an average velocity (X, Y or Z) generally constant throughout, so the sharp transition in velocity represents a change in structure. It should be appreciated that the above two described techniques may be used individually or in combination, depending on the particular implementation at hand.

The aforementioned embodiments thus provide discerning of specific structures by their velocities of motion. This provides superior differentiation between various structures observed during M-mode imaging. Additional features may also be provided or supplemented, as will be described in greater detail below.

According to one aspect of the present invention, differentiating step 220 may apply a velocity based color scale (a linear or non-linear color scale) to the M-mode data set extracted in step 200. More specifically, this aspect combines displayed movement via color (similar to color Doppler displays) with the spatial display of conventional M-mode to provide the user with a visual display (e.g., on display 110) of motion along the M-mode line of interest. Further information may be encoded into this display for some applications, such as varying the velocity based color scale in accordance with at least one of a direction of motion and a direction of a line along which the M-mode extraction (step 200) occurs. By way of example, motion towards the ultrasound transducer (part of equipment 150) may be displayed as differing shades of red (the shade depending on the magnitude of motion), whereas motion away from the ultrasound transducer may be displayed as differing shades of blue. In this manner, the user may be provided with a greater amount of information than in conventional M-mode displays, while maintaining a display format that is relatively easy for the user to interpret and understand.

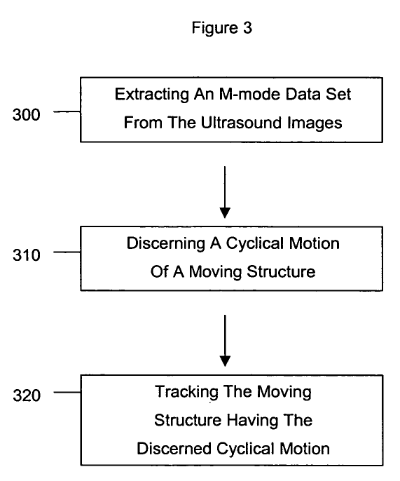

According to an embodiment of the present invention as shown in FIG. 3, a moving structure may be tracked on the basis of the velocity thereof. Tracking the position of the structure in an M-mode like representation may use a pixel pattern matching/searching algorithm either independently or as a function of the cardiac cycle as denoted by the electrocardiogram while using threshold and error/pixel variance margins to ascertain position of tracked anatomical structure.

By way of example, in step 300 the imaging system extracts an M-mode data set from a plurality of ultrasound images (similar to step 200), and then discerns a cyclical motion of a moving structure in step 310. By way of example, the imaging system may discern velocities as previously described in step 210, and store the discerned velocities in a memory file (e.g., a database). The imaging system may then examine the memory file over time to identify velocities that repeat over time, such as velocities that may correspond to a heartbeat. Other techniques for identifying cyclical motion in step 310 may also be used.

Once the cyclical motion has been discerned in step 310, the moving structure having the discerned cyclical motion may be tracked in step 320. According to one aspect of the present invention, tracking step 320 may be performed in real-time, though it may also be performed retrospectively from a database of stored ultrasound images or M-mode data sets. Tracking in step 320 may include highlighting on display 110 any abnormal motion (e.g., a delayed change in velocity, a change in the magnitude of velocity, etc.) for a user. This provides the user with a greater ability to identify problematic behavior of various structures observable using ultrasound.

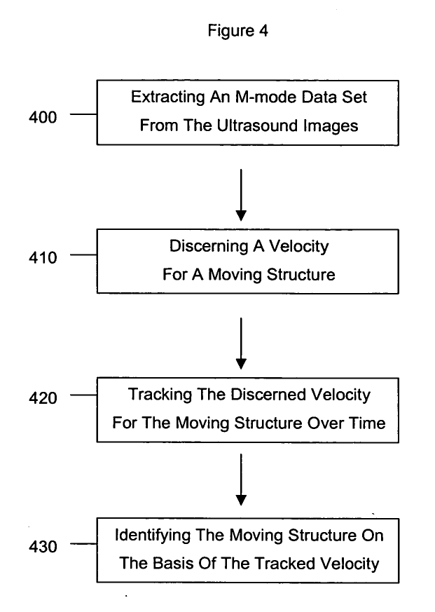

According to another embodiment of the present invention as shown in FIG. 4, the tracked velocity may be used to identify a given structure (possibly in addition to identifying abnormal movement by the identified structure). More specifically, in step 400 the M-mode data set is extracted from a plurality of ultrasound images, which is used in step 410 to discern a velocity for the moving structure. The imaging system continues to discern and store velocities for the moving structure, thereby “tracking” the velocity, or in effect, the structure, in step 420 for the moving structure over time. Preferably, tracking step 420 stores the discerned velocities over a period of time.

In step 430, the imaging system identifies the moving structure on the basis of the tracked velocity. By way of example, the imaging system may include a table of known motion characteristics for particular structures. Such a table may include, for example, ranges of commonly observed velocities and/or cyclical behavior for structures of interest (e.g., valves, heart wall, blood cells, etc.). This could also include correlations with the electrocardiogram. The tracked velocity of step 420 can then be compared in step 430 to this table, so as to identify the structure being tracked in step 420. In this manner, the imaging system may be able to identify structures observed during imaging. Other techniques for performing step 430 are also contemplated.

According to another embodiment of the present invention, the velocity information discerned in step 210 may be displayed along with supplemental data, such as corresponding electrocardiograph (ECG) data. By way of example, a structure of interest (e.g., a heart wall) may be selected, such as a user highlighting a discerned structure via interface 120. An ECG data source outputting ECG data may be used by the imaging system, such that the imaging system may then project on display 110 the velocity data discerned in step 210 along with corresponding ECG data for that structure. The present invention thus may be used to provide supplemental data (e.g., the ECG data), along with time/position data (from M-mode processing) and/or velocity data (discerned in step 210), thereby further giving the user a greater degree of data for the diagnosis and treatment of the patient.

According to yet another embodiment of the present invention, the present invention may be used to better identify and display structures of interest. By way of example, typical relational velocities may be used to narrow the field of possible structures prior to identification. Using the prior heart wall example, if the third group of data points have a zero velocity, then the imaging system may determine this region is outside of the heart. It may then presume that moving structures adjacent to this region can only include non-moving structures. Hence, the heart wall is one such structure that is adjacent to the region outside of the heart, whereas other moving structures (e.g. moving blood cells) are not. In this manner, the imaging system may determine that the second region is the heart wall, and identify it as such on the display 110.

According to yet another embodiment of the present invention, imaged structures may also be displayed (e.g., on display 110) by calculating the velocities of structures (to be displayed) along the line of interest using the Doppler processing techniques previously described, calculating relative backscattered powers and assigning lower backscattered powers to that of blood/fluids and higher backscattered powers to those from tissue, and calculating the relative size of the structure with the extents of the structure being either demarcated by the user using a pointing device or by defining a peak backscatter level and an associated power fall off at both ends along the M-mode line of interest to identify specific structures. Utilizing the relative velocities as well as backscattered power from structures, a color presentation can be presented and controlled either by the user or pre-set. Other display techniques are also contemplated.

According to another embodiment of the present invention, the present invention can be used to compensate for the angle of the chosen line with respect to transducer orientation. In a first step, the system obtains the coordinates of the points that comprise the M-mode line with respect to the B-mode image. The system then calculates the angle at each point along the line with respect to the ultrasound image line, and obtains the velocity and color information for each pixel along the line of interest. The velocity color scale previously described may then be applied for each point after accounting for the calculated difference in angle.

The present embodiment is useful in applications such as for ventricular lead placement, where accurate measurement of left ventricular function is required while the position of the leads to be placed are moved in search of an ideal position for these leads such that ventricular function (cardiac output) is maximized. M-mode offers a precise way of measuring the variation of distance between the free wall and the septum of the ventricle. However, the left ventricle is not always in a position to be imaged using M-mode given position of the catheter as well as the presence of other leads and catheters. Further, it is often ideal to track the position of the free wall as well as the septum as a function of time and the EKG to assess cardiac output in real time, thus allowing for hands-free operation of the ultrasonic imager during such procedures. Also, the velocity of the free wall as a function of time is of interest. In such cases, the velocity compensated for angle along the M-mode line is of greater relevance than the range of velocities that might present themselves, especially when using a phased array scan. Other applications are also contemplated.

The foregoing description of various embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principles of the invention and its practical application to enable one skilled in the art to utilize the invention in various embodiments and with various modifications as are suited to the particular use contemplated.

What is claimed is:

1. A method of processing ultrasound images, comprising:
extracting an M-mode data set from the ultrasound images;
discerning, from the M-mode data set, a first velocity for a first moving structure and a second velocity for a second moving structure, the second velocity being different from the first velocity; and
differentiating between the first moving structure and the second moving structure based on the discerned velocities.

2. The method of claim 1, wherein differentiating between the first moving structure and the second moving structure comprises applying a velocity based color scale to the M-mode data set.

3. The method of claim 2, wherein differentiating between the first moving structure and the second moving structure further comprises varying the velocity based color scale in accordance with at least one of a direction of motion and a direction of a line along which the M-mode extraction occurs.

4. The method of claim 2, further comprising displaying at least a portion of the M-mode data set with applied velocity based color scale as a color Doppler image.

5. The method of claim 1, wherein differentiating between the first moving structure and the second moving structure comprises:
tracking movement of at least one of the first moving structure and the second moving structure; and
discerning a cyclical motion of the tracked moving structure(s).

6. The method of claim 1, further comprising tracking a distance between the first moving structure and the second moving structure.

7. The method of claim 1, further comprising displaying at least one of the first velocity and the second velocity along with corresponding electrocardiograph (ECG) data.

8. The method of claim 1, further comprising identifying at least one of the first moving object and the second moving object.

9. The method of claim 1, further comprising displaying at least one of the first moving object and the second moving object.

10. A method of processing ultrasound images, comprising:
extracting an M-mode data set from the ultrasound images;
discerning a cyclical motion of a moving structure; and
tracking the moving structure having the discerned cyclical motion.

11. The method of claim 10, further comprising:
discerning, from the M-mode data set, a velocity for the moving structure.

12. A method of processing ultrasound images, comprising:
extracting an M-mode data set from the ultrasound images;
discerning, from the M-mode data set, a velocity for a moving structure;
tracking the discerned velocity for the moving structure over time; and
identifying the moving structure on the basis of the tracked velocity.

13. An ultrasound imaging device, comprising:
an interface adapted and configured to receive ultrasound image data; and
a controller adapted and configured to:
extract an M-mode data set from the ultrasound image data;
discern, from the M-mode data set, a first velocity for a first moving structure and a second velocity for a second moving structure, the second velocity being different from the first velocity; and
differentiate between the first moving structure and the second moving structure based on the discerned velocities.

14. The ultrasound imaging device of claim 13, wherein differentiating between the first moving structure and the second moving structure comprises applying a velocity based color scale to the M-mode data set.

15. The ultrasound imaging device of claim 14, wherein differentiating between the first moving structure and the second moving structure further comprises varying the velocity based color scale in accordance with at least one of a direction of motion and a direction of a line along which the M-mode extraction occurs.

16. The ultrasound imaging device of claim 14, wherein the controller is further adapted and configured to display at least a portion of the M-mode data set with applied velocity based color scale as a color Doppler image.

17. The ultrasound imaging device of claim 13, wherein differentiating between the first moving structure and the second moving structure comprises:
tracking movement of at least one of the first moving structure and the second moving structure; and
discerning a cyclical motion of the tracked moving structure(s).

18. The ultrasound imaging device of claim 13, wherein the controller is further adapted and configured to track a distance between the first moving structure and the second moving structure.

19. The ultrasound imaging device of claim 13, wherein the controller is further adapted and configured to display at least one of the first velocity and the second velocity along with corresponding electrocardiograph (ECG) data.

20. The ultrasound imaging device of claim 13, wherein the controller is further adapted and configured to identify at least one of the first moving object and the second moving object.

21. The ultrasound imaging device of claim 13, wherein the controller is further adapted and configured to display at least one of the first moving object and the second moving object.

22. An ultrasound imaging system, comprising:
means for acquiring ultrasound images of a region;
means for extracting an M-mode data set from the ultrasound images; and
means for differentiating between a first moving structure and a second moving structure in the region based on differences between the velocity and/or direction of motion of the first moving structure and the second moving structure.
